(19) United States
(12) Patent Application Publication
(10) Pub. No.: US 2026/0315772 A1
(43) Pub. Date: Oct. 15, 2026

(54) STERILE, DRY CAPS FOR MEDICAL MALE LUER FITTINGS AND METHODS FOR MAKING AND USING USE

(71) Applicant: CleanSite Medical, Inc., Solana Beach, CA (US)

(72) Inventors: John GRANT, Solana Beach, CA (US); Rick PLUTH, San Diego, CA (US); Daniel M. CHAMBERS, Solana Beach, CA (US)

(73) Assignee: CleanSite Medical, Inc., Solana Beach, CA (US)

(21) Appl. No.: 19/647,195

(22) Filed: Apr. 14, 2026

Related U.S. Application Data
(60) Provisional application No. 63/788,151, filed on Apr. 14, 2025.

(51) Int. Cl. A61M 39/20

(52) U.S. Cl. CPC A61M 39/20

(57) ABSTRACT

This invention provide articles, namely dry devices and systems, to protect male luer fittings of medical devices used for IV therapy, as well as methods of making and using such articles. The male luer fittings of such medical devices (e.g., syringes, catheter hubs, etc.) include tapered male protrusions with a central fluid flow path. In general, the dry devices of the invention comprise a (i) housing that provides at least a first cavity defined by a first side wall that encloses the first cavity and an opening defined by a circumferential sealing flange disposed at a portion of the first side wall proximate to the opening, and a closed end opposite the opening and (ii) a removable seal attached to the housing to prevent access to the first cavity while the seal is attached to the circumferential sealing flange.

### RELATED APPLICATION

This patent application claims the benefit and priority to U.S. provisional patent application Ser. No. 67/788,151, filed on 14 Apr. 2025. The aforementioned priority application is hereby incorporated by reference in its entirety for any and all purposes.

### TECHNICAL FIELD

The invention relates to medical devices useful in treating patients receiving IV therapy, particularly those receiving IV therapy.

### BACKGROUND

Luer fittings are widely used in the medical field, particularly to connect various components of IV lines in order to deliver various fluids to patients, including fluids used for hydration, nutrition, delivering medication, blood, and blood products, and the like. Such fittings can also be used to remove blood from patients. Today, IV lines, hemodialysis ports, chemotherapy ports, and the like employ luer fittings, which are ANSI-standardized fluid fittings for medical applications, ports, and interfaces used for making fluid-tight connections between various medical devices designed to deliver and remove fluids from patients. For example, some male luers include a tapered male protrusion that defines a lumen, where the protrusion extends out from the base having a central fluid opening and a sleeve or chamber spaced from the tapered male protrusion that has internal threads on its inner first wall. A luer lock or other female port with or without an external thread can be fitted into the sleeve and over the male protrusion to provide a friction-based fitting on the male protrusion, and if threads are present on a corresponding portion of the luer lock or female port, a threaded connection can also be made. A male luer can be used on syringes, injection ports, or other intravenous (IV) lines.

Today, several caps that contain a disinfecting solution of 70% isopropyl alcohol and water are available to clean and protect male luer protrusions used in IV therapy. Even so, further solutions are desired in the art due to cost concerns, variable efficacy, alcohol ingress into fluid flow paths, etc.

### SUMMARY OF THE INVENTION

The object of this invention is to address such concerns and provide articles, namely dry devices and systems, to protect male luer fittings of medical device used for IV therapy from microbial contamination, inorganic matter, blood and other bodily fluids, etc. Such fittings include the tapered protrusions of male luer fittings of medical devices having a central fluid flow path. Such fittings include syringes, catheter hubs, and the like.

Accordingly, one aspect of the invention concerns dry devices configured to protect a male luer fitting of a medical device used for IV therapy. In general, the dry devices of the invention comprise a (i) housing that provides a first cavity defined by a first wall that encloses the first cavity and an opening defined by a circumferential sealing flange disposed at a portion of the first wall proximate to the opening, and a closed end opposite the opening, and (ii) a removable seal attached to the housing's circumferential sealing flange to prevent access to the first cavity while the seal is attached to the circumferential sealing flange.

In some embodiments, the device may also contain foam or another matrix configured to contact and scrub or physically remove organic and/or inorganic matter from a surface of a male luer fitting of a medical device used for IV therapy, particularly the tapered male protrusion of such a fitting. In the context of the invention “dry” means that, once sealed with a removable seal, the interior of the device does not include liquid, for example, a liquid disinfectant such as 70% isopropyl alcohol (IPA).

In some embodiments, the device is configured to thread onto a male luer fitting by engaging complementary threads on the interior surface of the fitting's hub. In other embodiments, the device is of the invention is configured for a friction (or compression or pressure) fit.

The devices of the invention are advantageously sterilized after attaching the removable seal to the housing so as to close the cavity until the seal is removed, typically by a healthcare practitioner (e.g., a nurse) just before attaching the device to a male luer fitting of a medical device in order to protect that fitting, particularly its tapered male protrusion, from exposure to a healthcare environment, for example, the environment found in an ICU (intensive care unit) or general ward of an acute care hospital. As is known in the art, exposure of medical devices to healthcare environments often results in contamination of medical devices by microbes and other pathogens, bodily fluids (e.g., blood, urine), feces, inorganic material, and other contaminants.

In some embodiments, the housings of devices of the invention further comprise one or more threads or thread tabs extending out from an outer surface of the housing proximate the opening of the housing.

In some embodiments, the housings of devices of the invention further comprise one or more ridges extending out from an outer surface of the housing near the distal end of the housing opposite the opening.

In some embodiments, the housings of the invention comprise a first cavity, which cavity is advantageously cylindrical, advantageously a tapered cylinder having diameters that slightly decrease moving away from the opening toward the closed end of the cavity.

In some embodiments, the housing further comprises a smaller second cavity disposed in and substantially concentric with the first cavity. The second cavity is defined by a second wall that extends into the first cavity from the closed end of the housing, which second wall is shorter than the first wall. The second cavity has a second opening that is substantially concentric with the first opening and the inner surface of the second wall is configured to matingly engage an outer surface of the tapered male protrusion of the male luer fitting. The space between the first and second walls that form the first and second cavities is advantageously sized to at least accommodate the threaded hub of a male luer fitting. Advantageously, in some of these embodiments an outer surface of the second wall further comprises one or more threads or thread tabs extending out from the outer surface of the second wall proximate to the second opening. These threads or thread tabs are configured to engage complementary threads on the inner surface of the hub of the male luer fitting of the medical device to which the device of the invention is removably attached.

In some of the embodiments of the invention where the housing includes first and second cavities, the space between the first and second walls that form the first and second cavities is advantageously sized to accommodate not just the threaded hub of a male luer fitting but also a plurality of ribs (or other protrusions) from the inner surface of first wall to ensure a secure yet removable attachment of the housing to a male luer fitting of a medical device.

In some of the embodiments of the invention, the housing of the device further includes a tapered projection that extends into the first or second cavity from the closed end of the housing. The tapered projection is advantageously substantially concentric with the first (and, if present, the second) cavity, and is configured to sealingly engage a fluid exit of the male luer fitting when the housing is removably and securely attached to the medical device. In some of the embodiments, the tapered projection is telescopic, in that its length can adjust after contact with the fluid opening of the male luer fitting as the device of the invention is attached to the male luer fitting.

In some of the embodiments of the invention, the foam or another suitable scrubbing matrix is disposed in the first and/or second cavity in a manner that allows the foam or other matrix to contact, for example, the outer surface of the tapered male protrusion of a male luer fitting or the threaded region on the inner surface of the male luer fitting's hub.

As described, the devices of the invention are dry, in that they contain no liquid disinfectant and are not configured for use with disinfecting UV light. They can, however, contain disinfecting reagents (e.g., silver ions) blended into the materials used to manufacture the devices and/or one or more coatings applied during manufacture to a surface of the device intended to contact an exterior surface of a male luer fitting of a medical device that may become contaminated in a healthcare environment.

Another aspect of the invention concerns systems that include two or more, e.g., 2, 3, 4, 5, 6, 7, 8, 9, 10, 11-20 (inclusive), or more dry devices of the invention each configured to protect a male luer fitting of a medical device used for IV therapy. Such systems generally comprise a plurality of dry devices of the invention each configured to protect a male luer fitting of a medical device used for IV therapy. Such systems typically comprise a plurality of housings of the devices of the invention each removably attached to a strip of seal material (or lidding stock). In this way, a plurality of devices of the invention are sealed to a single piece of lidding material during manufacture, prior to sterilization. The lidding material advantagously includes a hole or slot at one or both ends to allow the system to be hung from a hook or other hanger on or near an IV pole at a patient's bedside. In this way, a plurality of devices of the invention can be made readily available to healthcare practitioners caring for the patient.

Such systems are advantageously sterilized after attaching the housings to a removable seal so as to close the cavities of the several housings until a particular housing is removed from the system, typically just before the housing is attached to a male luer fitting of a medical device in order to protect it.

In some embodiments, each of the housings further includes one or more threads or thread tabs extending out from an outer surface of the housing proximate the opening of the housing.

In some embodiments, each of the housings further includes one or more ridges extending out from an outer surface of the housing near the distal end of the housing opposite the opening.

In some embodiments having multiple cavities, the first cavity of each of the housings is cylindrical, advantageously a tapered cylinder having diameters that decrease moving away from the opening, toward the closed end of the cavity.

A related aspect of the invention concerns assemblies that include a housing of a device according to the invention that is removably attached to a male luer fitting of a medical device used for IV therapy, as would occur, for example, when a housing of a device of the invention is separated from its seal by a nurse who then places the unsealed device on a male luer fitting.

Yet another related aspect of the invention concerns methods of protecting a male luer fitting of a medical device used for IV therapy. Such methods typically involve attaching a housing of a device of the invention to a medical device having a male luer fitting after removing the removable seal from the housing (or removing one of plurality of housings sealed to a strip of seal material).

### BRIEF DESCRIPTION OF THE DRAWINGS

These and other aspects will now be described in detail with reference to the following drawings. Like reference symbols in the various drawings indicate like elements.

FIGS. 1 and 3 each show a cross section view of the housing portion of a dry cap according to the invention configured for a friction (or compression) fit to a male protrusion of a threaded male luer taper of a medical fitting. A removable seal attached to the housing's circumferential sealing flange is not shown. FIG. 8 shows a related embodiment of the device shown in FIG. 3 that also includes a scrubbing matrix and tapered projection.

FIG. 2 is a cross section view of the housing portion of a dry cap according to the invention configured for threaded attachment to a male protrusion of a threaded male luer taper of a medical fitting. A removable seal attached to the housing's circumferential sealing flange is not shown. FIG. 7 shows a related embodiment of the device shown in FIG. 2 that also includes a scrubbing matrix.

FIG. 4 is a cross section view of a dry cap according to the invention having concentric first and second cavities and configured for a friction (or compression) fit to the male protrusion of a threaded male luer taper of a medical fitting. Also shown are 5 such caps sealed to a single piece of lidding material configured to hang, for example, from an IV pole.

FIG. 5 is a cross section view of another alternative dry cap according to the invention having concentric first and second cavities and configured for threaded fitment to complementary threads on the inner surface of a hub of a medical fitting having a male luer taper. Also shown are 5 such caps sealed to a single piece of lidding material configured to hang, for example, from an IV pole.

FIG. 6 is a cross section view of an alternative dry cap according to the invention having concentric first and second cavities and configured for a friction (or compression) fit to the male protrusion of a threaded male luer taper of a medical fitting. Also shown are 5 such caps sealed to a single piece of lidding material configured to hang, for example, from an IV pole.

The details of one or more embodiments are set forth in the accompanying drawings and the description below. Other features and advantages will be apparent from the description and drawings, and from the claims.

### DETAILED DESCRIPTION

The specification, the attached drawings, and the appended claims describe articles, namely dry devices and systems, to protect male luer fittings of medical device used for IV therapy. Often, medical devices with male luer fittings include a tapered male protrusion protruding from an inner cavity of a hub, for example, a syringe hub or catheter hub, and extending beyond an opening to the hub for attachment of complementary vascular access devices such as needlefree connectors, IV administration sets, extension sets, and the like. Such tapered protrusions of male luer fittings typically have a central fluid flow path. The dry devices of the invention are configured to protect such tapered protrusions of male luer fittings used for IV therapy. In general, the dry devices of the invention comprise a housing that provides one or more substantially concentric open cavities defined by side walls that extend from a distal closed end wall to define the cavity(ies). The outermost wall is the longest (or tallest) and its lower opening, opposite the distal closed end wall from which the side wall(s) extends, defines a circumferential sealing flange to which a lid or seal can be attached (e.g., by heating sealing). The distal closed end is opposite the opening(s) of the cavity(ies). A removable seal is attached to the housing's circumferential sealing flange to prevent access to the cavity(ies) in the housing while the seal is attached to the circumferential sealing flange. Devices may be sealed individually or several may be sealed to a single larger piece of lidding material. The outer surface of the outermost side wall of a device of the invention can have one or more ribs or ridges, flanges, tabs, etc. to facilitate a user gripping the device with her or his fingers, for example.

In some embodiments, the interior surface of the housing's closed end can be flat while in some embodiments the closed end can include a tapered projection that extends into the cavity. Advantageously, the tapered projection is substantially concentric with the cavity and is configured to sealingly engage a fluid exit of the male luer fitting when the device is removably attached to the medical device. When so attached, the tapered projection seals the fluid exit of the medical fitting, thereby preventing contamination from entering the fluid path and associated surfaces in the medical fitting's lumen. In some embodiments, the length of the tapered projection is variable, in that its length can change, typically by reducing in length as the device is pushed or threaded onto a male luer fitting to cap and protect it. The taper3ed projection can be a single part or be two or parts designed to provide a telescoping configuration.

The housing of a device of the invention can be made from any suitable material (or combination of materials), advantageously plastic materials suitable for injection molding or other cost-effective production processes amenable to large-scale, automated manufacturing and device assembly. The housing and its various components are preferably formed as a single, integral unit during manufacturing (e.g., by injection molding). The housings can be manufactured by any suitable process, including extrusion, injection molding, and additive manufacturing (e.g., 3D printing).

In some embodiments, a dry device of the invention also includes foam or another suitable, preferably compressible, scrubbing matrix disposed in one or more device cavities and being configured to provide a scrubbing functionality as the device is attached or removed, or after attachment but before removal, to and/or from a male luer fitting. In most embodiments, the foam or scrubbing matrix is disposed in a cavity in the housing. The foam or scrubbing matrix is advantageously connected to the housing, for example, to the inner or outer surface of side wall of a second cavity and/or the inner surface of the side wall of a first cavity. Any suitable retaining configuration can be employed so as to allow the compressible foam or scrubbing matrix to scrub or otherwise clean, cleanse, or disinfect the surface(s) of the interfacing surface(s) of the male luer fitting. As will be appreciated, the compressible foam or scrubbing matrix is positioned in the device of the invention so as to contact one or more exterior surface(s) of a male luer fitting, particularly a male luer protrusion of such a fitting, when the fitting is connected to the device of the invention. The foam or scrubbing matrix, for example, an open-cell or felted foam, is preferably retained in the housing by one or more retaining elements, which element(s) assist in retention of the foam or scrubbing matrix in the housing in addition to transmitting rotational forces from the housing to the foam or scrubbing matrix as occurs during a procedure to disinfect or cleanse male luer connector. See, e.g., U.S. Pat. No. 11,065,431, hereby incorporated by reference, for a discussion of foams and scrubbing matrices suitable for use in the context of this invention.

Because the surface(s) to be cleansed may be contaminated with microorganisms that may form a biofilm (i.e., a matrix of microorganisms and extracellular material attached to a surface, which enables the microorganisms, typically bacteria and/or fungi, to adhere to a surface and carry out certain biochemical processes), the foam or scrubbing matrix also preferably has sufficient mechanical integrity when compressed and rotated to allow it to disrupt any biofilm that may be present on the surface of the male luer fitting, as can occur by rotating, twisting, or otherwise moving the foam or scrubbing matrix in relation to the male luer fitting, for example, by rotating the housing (to which the foam or scrubbing matrix is attached) in relation to the medical fitting. The resulting friction between the foam or scrubbing matrix and surface of the male luer fitting disrupts the biofilm, thereby cleansing the male luer fitting. Leaving the device secured to (i.e., capping) the fitting after such a cleansing operation will limit, and preferably preclude, biofilm regrowth and/or the microbial recolonization of cleansed surfaces (which remain in contact with the compressible foam or scrubbing matrix).

In preferred embodiments, the compressible foam or scrubbing matrix includes one or more dry cleansing reagent species dispersed therein, preferably at the time the device is manufactured. Such dry or non-liquid microbial disinfecting agents suitable for medical foams include quaternary ammonium compounds, polyhexamethylene biguanide (PHMB), triclosan, and solid oxidizing agents like silver compounds or copper oxide. These agents can be incorporated into polymeric foam structures during manufacturing to provide sustained antimicrobial release. As those in the art will appreciate, such agents will be selected for their ability to be incorporated into materials, solid form stability, and broad-spectrum activity against bacteria and fungi so as to be suitable for surface disinfecting foam applications.

In some preferred embodiments, the compressible foam or scrubbing matrix includes two or more components. In some of such embodiments, one component is attached to the inner surface of the side wall of the first cavity, another component is secured to the outer surface of side wall of the first cavity, and another component is secured to the inner surface of the side wall of the second cavity. In some embodiments, the component(s) of the compressible foam or scrubbing matrix secured to the housing is(are) preferably configured to radially compress when brought into contact with a male luer fitting to be capped and potentially cleansed.

The devices of the invention also include a removable lid or seal to separate the interior spaces and structures of the device from the external environment. Such a lid or seal prevents exposure of the device's interior to the environment until the seal is removed, typically by a healthcare worker just prior to her/his use of the device to cap and, if desired and if the device is so configured, to clean/cleanse male luer fitting to which it is connected. The removable seal (or strip of a or seal material) is attached to the housing (or a plurality of housings) during manufacture, advantageously before sterilization. After sterilization by any suitable method (e.g., irradiation (e.g., gamma or e-beam irradiation), exposure to ethylene oxide, etc.), the seal maintains the sterility of the interior portions of the housing until the seal is removed (or a device is removed from a lidding strip to which several housings are sealed) by a healthcare practitioner, typically just before the housing is removably attached to a medical device with a male luer fitting. The removable seal can be a foil-based seal. Devices can be individually sealed single units or they two or more housings can be sealed on a single piece of lidding material using any suitable process, including heat-sealing, thermal bonding, gluing, etc.

### Representative Embodiments

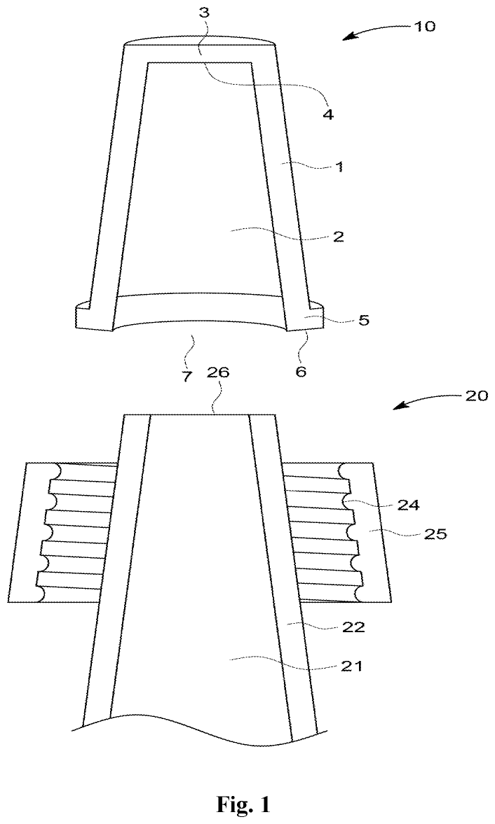

To further illustrate and describe certain preferred, representative embodiments of the invention, the reader is directed to the appended drawings, FIGS. 1-8, which illustrate various particularly preferred embodiments of the capping and cleansing devices of invention. A description of these preferred, representative embodiments follows.

FIG. 1 shows a cross section view of the housing portion (10) of a dry cap according to the invention configured for a friction (or compression) fit to a male protrusion (22) of a threaded male luer taper of a medical fitting (20). The housing (10) includes a cavity (2) defined by a side wall (1) that encloses the cavity (2) and an opening (7) defined by a circumferential sealing flange (5) disposed at a portion of the first wall proximate to the opening, and a closed end (3) opposite the opening from which the side wall (1) extends. The circumferential sealing flange (5) includes a sealing surface (6) to which the seal (not shown) is attached during manufacture, before the device is sterilized, for example, by gamma irradiation. After the seal is removed, the housing (10) can be attached by pressing it onto the male protrusion (22) of a threaded male luer taper of a medical fitting (20), here a threaded hub. The medical fitting includes a fluid channel (21), the exit of which is an opening (26) at the tapered tip of the medical fitting. The medical fitting also includes threads (24) on the inner surface of the hub's wall (25).

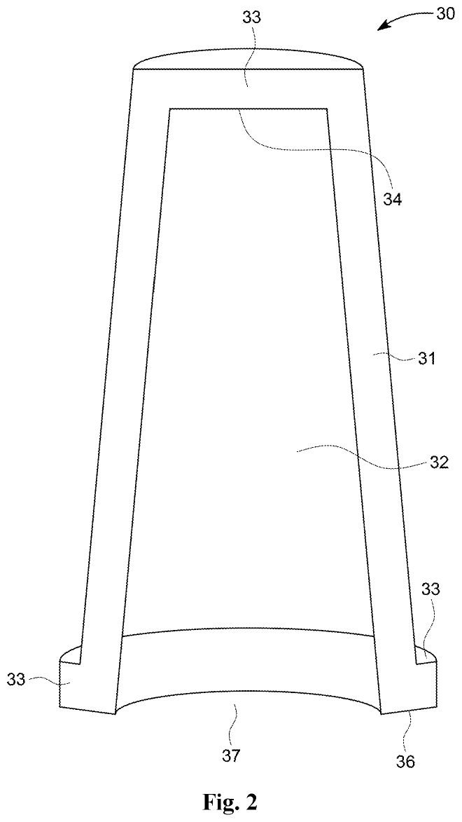

FIG. 2 is a cross section view of the housing portion (30) of a dry cap according to the invention configured for threaded fitment to a male protrusion of a threaded male luer taper of a medical fitting. A removable seal attached to the housing's circumferential sealing flange is not shown. Here, the housing (30) again includes a cavity (32) defined by a side wall (31) that encloses the cavity (22) and an opening (37) defined by a circumferential sealing flange (36) disposed at the bottom of the wall (31) proximate to the opening (37), and a closed end (33) opposite the opening (37) from which the side wall (31) extends. As with other embodiments of the invention, the interior surface of the housing's closed end (34) can be flat while in some embodiments the closed end can include a tapered projection (not shown) that extends into the cavity. Advantageously, the tapered projection is substantially concentric with the cavity and is configured to sealingly engage a fluid exit (see, e.g., 26, FIG. 1) of the male luer fitting when the device is removably attached to the medical device. When so attached, the tapered projection seals the fluid exit of the medical fitting, thereby preventing contamination from entering the fluid path and associated surfaces in the medical fitting's lumen. In the embodiment shown in FIG. 2, the housing (30) includes two thread tabs (33) disposed opposite one another at the lower edge of the wall (31) of the housing (30). These thread tabs can be used, for example, to engage complementary threads on a threaded hub of a medical fitting that includes a threaded male luer (see, e.g., 26, FIG. 1).

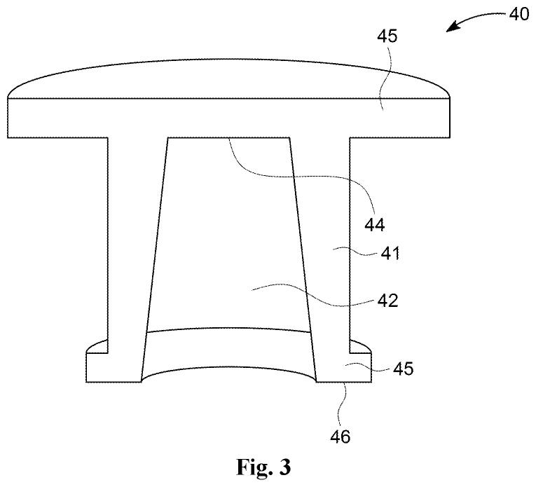

FIG. 3 shows a cross section view of another housing embodiment (40) of a dry cap according to the invention also configured for a friction (or compression) fit to a male protrusion of a threaded male luer taper of a medical fitting. A removable seal attached to the housing's circumferential sealing flange is not shown. The embodiment shown in this Figure is similar to the embodiment shown in FIG. 1, the primary difference being that in the embodiment shown in FIG. 3, the top of the housing (45) has a greater circumference than that defined by the outer surface of the side wall (41) that defines the cavity (42) of the housing (40). The larger top can facilitate attachment to a medical fitting with a male luer taper, particularly in embodiments (not shown) where the outer surface of the wall of the housing includes threads or thread tabs near the opening to the cavity. As shown, the housing includes a circumferential sealing flange (45), which includes a sealing surface (46) to which a seal (not shown) can be attached.

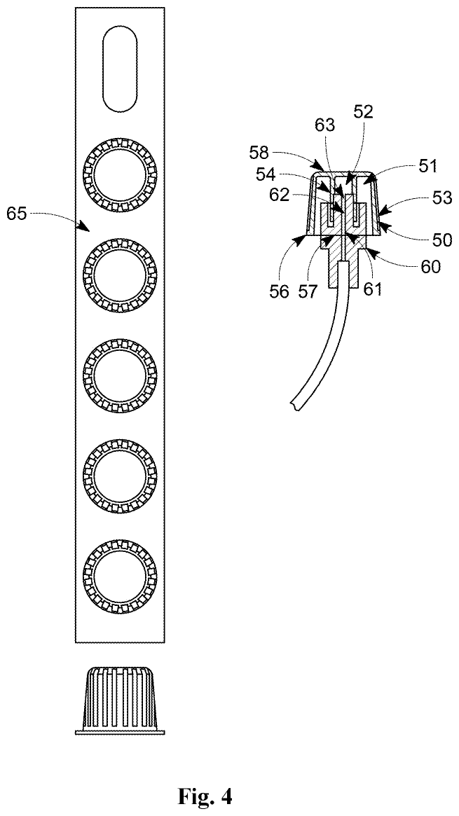

FIG. 4 is a cross section view of a dry cap according to the invention having concentric first and second cavities and configured for a friction (or compression) fit to the tapered male protrusion of a threaded male luer fitting (60). Also shown are 5 such caps sealed to a single piece of lidding material configured to hang, for example, from an IV pole. Here, the housing portion (50) of a dry cap is configured to attach to a tapered male protrusion (62) of a threaded male luer fitting (60). The housing (50) includes first and second cavities (51, 52) defined by a first and second side walls (53, 54) extending from the closed end (58) and an opening (57) opposite the closed end (58), which opening (57) is defined by a circumferential sealing flange (56) disposed at a portion of the first side wall (53) proximate to the opening (57), and a closed end (58) opposite the opening. The circumferential sealing flange (56) includes a sealing surface to which the seal (65) is attached during manufacture, before the device is sterilized, for example, by gamma irradiation. After a user pulls a housing (50) from the seal (65), the housing (50) can be attached by pressing it onto the tapered male protrusion (62) of a threaded male luer fitting (60). The medical fitting includes a fluid channel (61), the exit of which is an opening (63) at the tapered tip of the medical fitting (60).

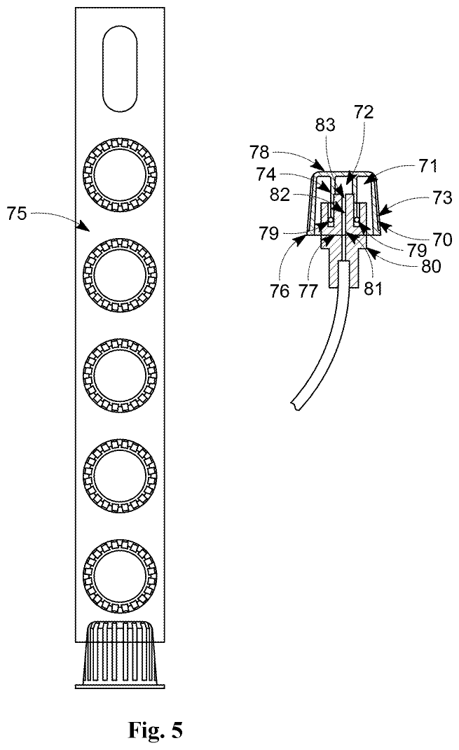

FIG. 5 is a cross section view of an alternative dry cap according to the invention having concentric first and second cavities (71, 72) but configured for threaded fitment to a tapered male protrusion (82) of a threaded male luer fitting (80). Also shown are 5 such caps (each, 70) sealed to a single piece of lidding material (75) configured to hang, for example, from an IV pole. Here, the housing portion (70) of a dry cap is configured to attach to a tapered male protrusion (82) of a threaded male luer fitting (80). The housing (70) includes first and second cavities (71, 72) defined by a first and second side walls (73, 74) and an opening (77) defined by a circumferential sealing flange (76) disposed at a portion of the first wall (73) proximate to the opening (77), and a closed end (78) opposite the opening. The space between the first and second walls (73, 74) that form the first and second cavities (71, 72) is advantageously sized to at least accommodate the threaded hub of the male luer fitting (80). Advantageously, in this embodiment an outer surface of the second wall further comprises one or more threads or thread tabs (79) extending out from the outer surface of the second wall (74) proximate to the second opening. These threads or thread tabs (79) are configured to engage complementary threads on the inner surface of the hub of the male luer fitting of the medical device (80) to which the device of the invention is removably attached. The circumferential sealing flange (76) includes a sealing surface to which the seal (75) is attached during manufacture, before the device is sterilized, for example, by gamma irradiation. After the seal is removed, the housing (70) can be attached by threading it onto the male protrusion (82) of a threaded male luer taper of a medical fitting (80). The medical fitting includes a fluid channel (81), the exit of which is an opening (83) at the tapered tip of the medical fitting (80).

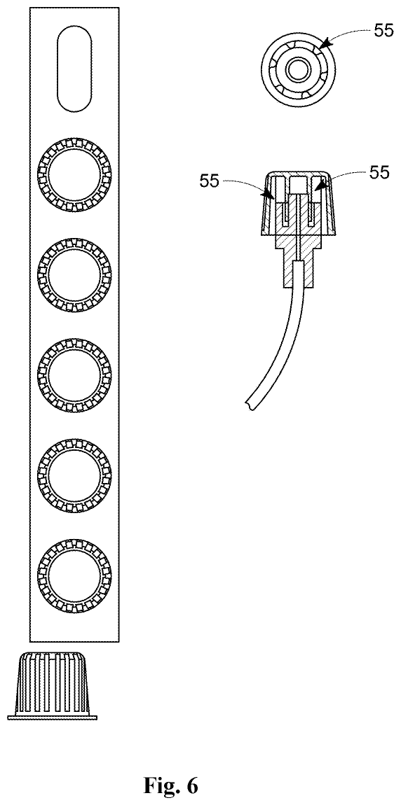

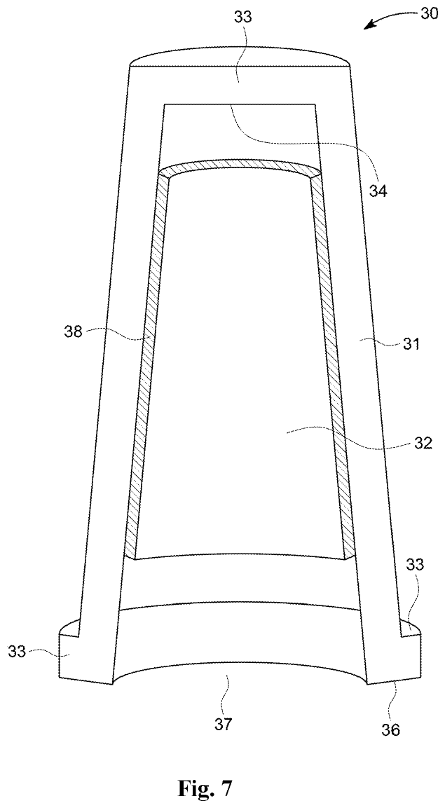

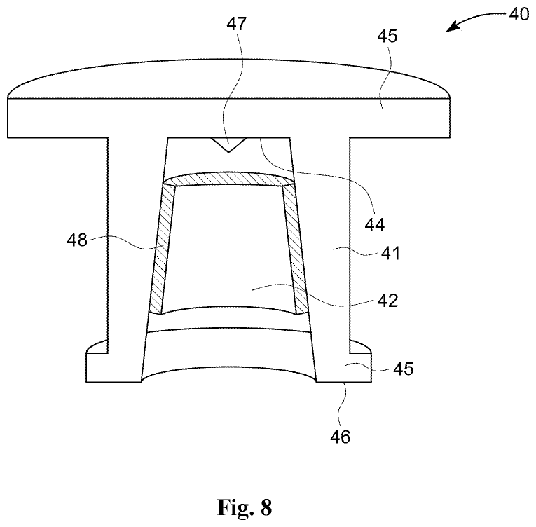

FIG. 6 is a cross section view of an alternative dry cap according to the invention that is analogous to the embodiment shown in FIG. 4, the difference being that in the embodiment of FIG. 6 the housing portion (50) of the dry cap includes 6 ribs (55) that protrude into the space between the first and second walls (53, 54) that form the first and second cavities (51, 52). These ribs (55) stabilize and help secure the housing by way of a compression fit when it is pressed onto the male luer fitting.

FIG. 7 shows a related embodiment of the device shown in FIG. 2 that also includes a scrubbing matrix (38) attached to the inner surface of the housing's side wall (31). Attachment can be by any suitable method (not shown), including mechanical features, adhesive, or the like. As shown, the scrubbing matrix (38) can cover a portion or all of the interior surface of the housing side wall (31).

FIG. 8 shows a related embodiment of the device shown in FIG. 3 that also includes a scrubbing matrix (48) and tapered projection (47). Attachment of a scrubbing matrix (48) can be by any suitable method (not shown), including mechanical features, adhesive, or the like. As shown, the scrubbing matrix (48) can cover a portion or all of the interior surface of the housing side wall (41). The device depicted in FIG. 8 also includes a tapered projection (47) extending from the underside of the top of the device (45) inward into the cavity (42). The tapered projection is advantageously substantially concentric with the side wall (41).

Unless the context clearly requires otherwise, throughout the description above and the appended claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list, and any combination of the items in the list.

The foregoing description, for purpose of explanation, has been described with reference to specific embodiments. However, the illustrative discussions above are not intended to be exhaustive or to limit the invention to the precise forms disclosed. Many modifications and variations are possible in view of the above descriptions. The embodiments were chosen and described in order to best explain the principles of the invention and its practical applications to thereby enable others skilled in the art to best utilize the invention and various embodiments with various modifications as are suited to the particular use contemplated. As such, the invention extends to all functionally equivalent structures, methods, and uses, such as are within the scope of the appended claims, and it is intended that the invention be limited only to the extent required by the applicable rules of law

1. A device configured to protect a male luer fitting of a medical device used for IV therapy, the device comprising:
a. a housing defining a first cavity defined by a first side wall that encloses the first cavity and an opening defined by a circumferential sealing flange disposed at a portion of the first side wall proximate to the opening, and a closed end opposite the opening; and
b. a removable seal attached to the circumferential sealing flange of the housing to prevent access to the first cavity while the seal is attached to the circumferential sealing flange,
wherein space defined by the first cavity is dry.

2. A device according to claim 1 that has been sterilized.

3. A device according to claim 1 wherein the housing further comprises one or more threads or thread tabs extending out from an outer surface of the housing proximate to the opening.

4. A device according to claim 1 wherein the housing further comprises one or more ridges extending out from an outer surface of the housing near the distal end of the housing opposite the opening.

6. A device according to claim 1 wherein the first cavity is cylindrical or a tapered cylinder.

7. A device according to claim 6 wherein the housing further comprises a smaller second cavity disposed in and substantially concentric with the first cavity, which second cavity is defined by a second wall that extends into the first cavity from the closed end of the housing and which is shorter than the first wall, the second cavity having a second opening that is substantially concentric with the first opening, wherein an inner surface of the second wall is configured to matingly engage an outer surface of the male luer fitting of the medical device.

8. A device according to claim 1 wherein an outer surface of the second wall further comprises one or more threads or thread tabs extending out from the outer surface of the second wall proximate to the second opening.

9. A device according to claim 1 that further comprises a tapered projection that extends into the first cavity from the closed end of the housing, is substantially concentric with the first cavity, and is configured to sealingly engage a fluid exit of the male luer fitting when the device is removably attached to the medical device.

10. An assembly comprising a housing of a device according to claim 1 removably attached to a male luer fitting of a medical device used for IV therapy.

11. A method of protecting a male luer fitting of a medical device used for IV therapy, comprising attaching a housing of a device according to claim 1 to the medical device after removing the removable seal from the housing.

12. A system that comprises a plurality of devices each configured to protect a male luer fitting of a medical device used for IV therapy, the system comprising a plurality of housings of the device according to claims each removably attached to a strip of seal material to prevent access to the first cavity of the housing while the seal is attached to the circumferential sealing flange of the housing, wherein each of the device does not contain a liquid disinfectant.

13. A system according to claim 12 that has been sterilized.

14. A method of protecting a male luer fitting of a medical device used for IV therapy, comprising attaching a housing from a system according to claim 12 to the medical device after removing the housing from the strip of seal material.
